SEQUENCE LISTING (8 sequences)

SEQ ID NO: 1 - DNA, 1122 bp, Artificial
  Synthetic DNA
atgcagaacagaaggaaagcgttttacaagctctgtcctaatgcaatgccggatttgtccaaccggttgagtaaaaagcagagtaaaactgagaagaagctgcacaagaatatatttgcacacactggcccagtgaccatctatgttgatccaccttccagtgcagtagactcagcacttgctacaatcgattggcaagatctggttgattgcaattctgtcattcagcaggacgccgctgggggcgcgattacacagcagcaggaccactgtctctctggggaacctgactttgacctgcaggaattccgagatgcagtcgatcagtttattgcagatcagccctctctcatgcagcccagtcttggccccccagaatttcagctcccagcctgcaacgtgccagtgtttgagccctgtatgaccaaccccctgcaagctcagccggagcacctgctgcctatcaatgtgcagaccattccctccacggagcaatactggagagatgtggctgatcacaaccagaaagcactcggggatgccttggtggagaacaaccagctccaggtgagtctgactgagaaacaggaggagatcgtttctctgaaggagaagaacatccagctaaatgaactggccaatcaagcaaagcatctgtcttctgtccttgataaactaatgaaagaaagaacaaagcaaaattccggggcaactcagggaagactcccagtgaaacgaagcctagaggacttttacccccagagcaatgagccggactctactcaggtggatgaaatactgcgggaaatctctaagaaatgcaatatagccttaatgggcagcgacctcagtgaacgcaagcgccccaggctggagcctatggactcaatggattggcaagaggaaggcgtaacggaaattaaaatgtgcggggcatttcacggactcaaaacatcgacagggctgaactctgtcaacttgggtgacacagatctagaagatgtctccttcaggacttccatcaaagagcacagcacaatcaggaccttggcatttccccaaggaaacgcctttaccattcggacttctggtgggggctacaaatttagatgggttcccaat

SEQ ID NO: 2 - protein, 374 aa, Artificial
  Synthetic polypeptide
MQNRRKAFYKLCPNAMPDLSNRLSKKQSKTEKKLHKNIFAHTGPVTIYVDPPSSAVDSALATIDWQDLVDCNSVIQQDAAGGAITQQQDHCLSGEPDFDLQEFRDAVDQFIADQPSLMQPSLGPPEFQLPACNVPVFEPCMTNPLQAQPEHLLPINVQTIPSTEQYWRDVADHNQKALGDALVENNQLQVSLTEKQEEIVSLKEKNIQLNELANQAKHLSSVLDKLMKERTKQNSGATQGRLPVKRSLEDFYPQSNEPDSTQVDEILREISKKCNIALMGSDLSERKRPRLEPMDSMDWQEEGVTEIKMCGAFHGLKTSTGLNSVNLGDTDLEDVSFRTSIKEHSTIRTLAFPQGNAFTIRTSGGGYKFRWVPN

SEQ ID NO: 3 - DNA, 2100 bp, Artificial
  Synthetic DNA
attggtactgtgacaggttgcagccctgggaggcgagctacagaaatcttctcttttctttaacacatctaacgagggtttcactccccaaacctcccaaaaaatgcagaacagaaggaaagcgttttacaagctctgtcctaatgcaatgccggatttgtccaaccggttgagtaaaaagcagagtaaaactgagaagaagctgcacaagaatatatttgcacacactggcccagtgaccatctatgttgatccaccttccagtgcagtagactcagcacttgctacaatcgattggcaagatctggttgattgcaattctgtcattcagcaggacgccgctgggggcgcgattacacagcagcaggaccactgtctctctggggaacctgactttgacctgcaggaattccgagatgcagtcgatcagtttattgcagatcagccctctctcatgcagcccagtcttggccccccagaatttcagctcccagcctgcaacgtgccagtgtttgagccctgtatgaccaaccccctgcaagctcagccggagcacctgctgcctatcaatgtgcagaccattccctccacggagcaatactggagagatgtggctgatcacaaccagaaagcactcggggatgccttggtggagaacaaccagctccaggtgagtctgactgagaaacaggaggagatcgtttctctgaaggagaagaacatccagctaaatgaactggccaatcaagcaaagcatctgtcttctgtccttgataaactaatgaaagaaagaacaaagcaaaattccggggcaactcagggaagactcccagtgaaacgaagcctagaggacttttacccccagagcaatgagccggactctactcaggtggatgaaatactgcgggaaatctctaagaaatgcaatatagccttaatgggcagcgacctcagtgaacgcaagcgccccaggctggagcctatggactcaatggattggcaagaggaaggcgtaacggaaattaaaatgtgcggggcatttcacggactcaaaacatcgacagggctgaactctgtcaacttgggtgacacagatctagaagatgtctccttcaggacttccatcaaagagcacagcacaatcaggaccttggcatttccccaaggaaacgcctttaccattcggacttctggtgggggctacaaatttagatgggttcccaattaagaatgacaacggtcagctaactacacttccactaatgtactcagaaattcttgccttgttttgaccttgcactccattgtgcagcatatctaacctgccactagagggcgcacaattacctctttttttcttttatttctgtgctaccggtactttaaatttctgccaaatgaatcctcaagtagaaaccagcaaaactgggaggctttccaaactttttttgtatttctttattggatatgaaggatatttgcattgctatgactataaatactgctatttacatttcaggacaataaggctggttatcagagataatctgatccattaaaaccactggtggctatgagttttcttgcattatgaaaaatgtagatgtccgttgtttgtaaactatacgttttcatttgcactttgattcaatgaaagaacattttgtttgaatgaacaaaggaaggacctgccttcacagtgttaattaaaaagcattttttaaagtccacaataagccattcaattcaaaaatagacacacaagatagcccatgagggccatgactgcactgaaaaagataacattccatttatcagctaaccaattcccatgtaataaatgtaataagtaacatttttggtttacaaaataacatataaattactataaaaaatctaatttgctacatttatattgttaatataagaatgtctttgttatttaaacacactgatttttatatttgttttgtttttttactgcggttcttttttgtatggaaacttttttttaaaattgttttttaagtaaaataatttatatgaaaaaaaaaaaaaaaaaaaaaaaaaaaaaaaaaaaaaaaaaaaaaaaaaaaaaaa

SEQ ID NO: 4 - protein, 392 aa, Artificial
  Synthetic polypeptide of majority consensus sequence
MQXXXXXXXXRXAFXXXCPNXMXDLSXRXXXKXXKXEXKXXXXXXXXXGXXXXXPVXXYXDPPXXXXAXXXALXTIDXQDLXDCXSXXXXXAXXXGXXXXXQXXXLXXEXDFXLQXFRDAXDXXIADXXSLMXPXLXXXXFXXXXCXVXXFXXCXXXXLXXXXXXXLXXXXXXXPXTEQYWXXVADXNQXALGXALXENNQLXVXLTXKQEEIXSLXEXNXQLXELAXXXXHLXSVLDKLMXXXXXXXXXXXQXXXXXKRSLEXXXXXXXXXXXXXXXVDXILRXISXXCXXALXXXDXXXXKRPRLXPXXXXXXXXXXXXXXXXXGAFXGLXTXXXXXSVNLXXXXLEXXXSFXTXIXXHSTIRTLAFPQGXAFTIRTXXGGYKFRWVPXI

SEQ ID NO: 5 - protein, 303 aa, Artificial
  Synthetic polypeptide of majority consensus sequence
MQACXGXAAGRRAFDSICPNRMLDLSXRXLXKPGKPERKFXPXXKXFXGCXGGSPVXVYEDPPDAEPXXLPALTTIDLQDLADCXSLLGXDAXPGGDXXASQNHSLQTEXDFNLQXFRDAVDDLIADSSSLMSPXLXXXDFPFSPCDVSXFGPCLSPPLDPXALXSPXLXXPPTEQYWKEVADQNQRALGDALVENNQLHVTLTQKQEEIASLKERNVQLKELASRTRHLASVLDKLMITQSXAEPFQXKAXXKRSLEELXXXXXXAGQXCAEVDAILREISXRCXEALXXRDPKRPRLXPEP

SEQ ID NO: 6 - protein, 379 aa, Artificial
  Synthetic polypeptide of hypothetical protein from LOC622408 of Mus musculus
MQACEGSAAGRRAFDSICPNRMLDLSRRTLGKPGKPERKFVPSWKSFSGCGGGSPVAVYEDPPDAEPAPLPALTTIDLQDLADCTSLLGTEASPSGDSSASQNPSLQTEEDFNLQNFRDAMDDLIADSSSLMSPPLTNSDFPFSPCDVSSFGSCLSPSLDPPALGSPDLPPPPTEQYWKEVADQNQRALGTALIENNQLHVTLTQKQEEIASLRERNVQLKELASRTRHLASVLDKLMITQSPAEPFQIKATTKRSLEELFCAAGQAGQGCAEVDAILRDISQRCEEALHNRDPKRPRLQPEPDSKDCSSRNLHGAFRGLRTDCSASSVNLSHSELEEGGSFSTPIRSHSTIRTLAFPQGKAFTIRTVTGGYKFRWVPS

SEQ ID NO: 7 - protein, 309 aa, Artificial
  Synthetic polypeptide of hypothetical protein from LOC345643 of Homo sapiens
MQACGGGAAGRRAFDSICPNRMLALPGRALLCKPGKPERKFAPPRKFFPGCTGGSPVSVYEDPPDAEPTALPALTTIDLQDLADCSSLLGSDAPPGGDLAASQNHSHQTEADFNLQDFRDTVDDLISDSSSMMSPTLASGDFPFSPCDISPFGPCLSPPLDPRALQSPPLRPPDVPPPEQYWKEVADQNQRALGDALVENNQLHVTLTQKQEEIASLKERNVQLKELASRTRHLASVLDKLMITQSRDCGAAAEPFLLKAKAKRSLEELVSAAGQDCAEVDAILREISERCDEALQSRDPKRPRLLPEP

SEQ ID NO: 8 - protein, 374 aa, Artificial; identical to SEQ ID NO: 2
  Synthetic polypeptide of hypothetic protein from bc124892 from Xenopus laevis

(19) United States
(12) Patent Application Publication
(10) Pub. No.: US 2013/0045537 A1
(43) Pub. Date: Feb. 21, 2013

(54) METHODS AND COMPOSITIONS RELATING TO MULTICILIATE CELL DIFFERENTIATION

(71) Applicants: Christopher Kintner, La Jolla, CA (US); Jennifer Stubbs, La Jolla, CA (US)

(72) Inventors: Christopher Kintner, La Jolla, CA (US); Jennifer Stubbs, La Jolla, CA (US)

(73) Assignee: The Salk Institute for Biological Studies, La Jolla, CA (US)

(21) Appl. No.: 13/580,645

(22) PCT Filed: Feb. 23, 2011
(86) PCT No.: PCT/US11/25896

§ 371 Date: Oct. 30, 2012

Related U.S. Application Data
(60) Provisional application No. 61/307,206, filed on Feb. 23, 2010.

(51) Int. Cl. C07K 14/435; C07K 14/47; C12N 5/10; C07K 16/18; C12N 15/12; C12N 15/85

(52) U.S. Cl. 435/455; 536/23.5; 435/320.1; 435/325; 530/350; 530/387.9

(57) ABSTRACT

Compositions and methods relating to development of multiciliate cells are provided.

### CROSS-REFERENCES TO RELATED APPLICATIONS

The present application claims priority to U.S. Provisional Appl. No. 61/307,206, filed Feb. 23, 2010, the disclosure of which in incorporated herein in its entirety.

### BACKGROUND OF THE INVENTION

Cilia are ancient microtubule-based organelles that project finger-like from the surface of eukaryotic cells (Marshall and Nonaka (2006) Curr Biol 16: p. R604-14). Various estimates suggest that over 600 different proteins are required to form cilia, and many of these are conserved throughout the animal and plant kingdoms where cilia are found (Inglis et al. (2006) Trends Genet. 22:491-500; Pazour (2004) Curr. Biol. 14:R575-77). Cilia have diversified in vertebrates, resulting in several subtypes generally classified as motile or non-motile (Ibanez-Tallon et al. (2003) Hum Mol Genet 12 Spec No 1:R27-35 and FIG. 1). Nonmotile cilia are used to detect light, mechanical and chemical stimuli, and range in subtype from the outer segment of photoreceptors to the primary cilium found on essentially all mammalian cells (Eggenschwiler and Anderson (2006) Annul Rev Dev Biol). Motile cilia subtypes range from the single cilium found on node cells and used for left-right patterning to the hundreds of cilia found on multiciliate cells (Hirokawa et al. (2006) Cell 125:33-45). Uncertainties remain as to the genes belonging to the ciliome, how the proteins they encode assemble into ciliate structures, or nanomachines, that comprise this organelle, how the various cilia subtypes are specified, and how this process is integrated into the mechanisms of cell type differentiation.

Multiciliate cells are specialized epithelial cells that use motile cilia to produce directed fluid flow in vertebrates. These cells produce robust fluid flow along luminal surfaces, using hundreds of motile cilia that project from their apical surface. Because fluid flow is useful physiologically for several reasons, multiciliate cells are employed in diverse organ systems, ranging from respiratory and reproductive tract to the ventricles of the brain (Ibanez-Tallon et al. (2003) Hum Mol Genet 12 Spec No 1:R27-35). The significance of these cells to organ formation and function is highlighted by disease states such as primary ciliary dyskinesia (PCD) and Kartegener's syndrome (Baker and Beales (2009) Am J Med Genet C Semin Med Genet 151C(4):281-95). In these celiopathies, the multiciliate cells located in the lung, middle ear, brain, reproductive tract and at the node are defective, resulting in recurring respiratory tract infections, otitis, hydrocephaly, infertility and situ inversus, respectively. Diagnosis and treatment of these human diseases would benefit from a better understanding of the genetic and developmental mechanisms that enable multiciliate cells to form and function.

One insight into the transcriptional code required for multiciliate cell differentiation comes from the analysis of FoxJ1, a member of the winged-helix family of transcription factors. First identified in the mouse based on expression in the lung, the loss of FoxJ1 by gene targeting leads to a profound absence of cilia in multiciliate cells in all tissues where they are found (Blatt et al. (1999) Am J Respir Cell Mol Biol 21:168−76; Brody et al. (1997) Genomics 45:509; Chen et al. (1998) J Clin Invest 102:1077-82). Only motile cilia are affected in FoxJ1 mutants, nonmotile cilia subtypes form normally (Brody et al. (2000) Am J Respir Cell Mol Biol 23:45-51; Gomperts et al. (2004) J Cell Sci 117:1329).

More recent studies in X. laevis and Zebrafish suggest that FoxJ1 is required to activate gene expression required for motile cilia formation, thereby acting relatively late in the differentiation of multiciliate cells (Stubbs et al. (2008) Nat Genet 40:1454; Yu et al. (2008) Nat Genet 40:1445). Thus, in FoxJ1 loss-of-function, multiciliate cells still initiate differentiation by forming hundreds of basal bodies but then cilia formation is blocked by a failure to dock these at the apical surface where cilia can form (see, e.g., FIG. 1; Gomparts; Huang et al. (2003) J Cell Sci 116:4935; Pan et al. (2007) J Cell Sci 120:1868). In FoxJ1 gain-of-function experiments, epithelial cells are induced to form motile cilia when FoxJ1 is ectopically expressed, but these cells resemble the monociliated cells involved in left-right patterning, not multiciliate cells.

The results described herein lead to the model shown in FIG. 1, where FoxJ1 can drive gene expression required for motile cilia, both in multiciliate and node-like cells. Prior to the present disclosure, the transcription factor that acts to drive the earlier steps in multiciliate cell differentiation, e.g., required for cell cycle arrest, basal body production, and the activation of FoxJ1 expression, was not known. The compositions and methods provided herein identify a protein of previously unknown function termed Multcilin (MCI) as a key regulator of ciliate cell differentiation.

### BRIEF SUMMARY OF THE INVENTION

Provided herein are methods and compositions for detecting and preparing ciliate cells, and diagnosing and treating ciliate cell related disorders. In some embodiments, the invention provides an isolated nucleic acid encoding a multicilin (MCI) protein, e.g., a Xenopus, mammalian, or human MCI protein. In some embodiments, the MCI protein comprises the amino acid sequence of SEQ ID NO:4 or 5. In some embodiments, the MCI protein comprises an amino acid sequence having at least 85, 90, 91, 92, 93, 94, 95, 96, 97, 98, 99, or 100% identity to a sequence selected from: at least 25, 30, 35, 40, 45, or 50 consecutive amino acids of SEQ ID NO:2; at least 25, 30, 35, 40, 45, or 50 consecutive amino acids of SEQ ID NO:6; and at least 25, 30, 35, 40, 45, or 50 amino acids of SEQ ID NO:7. In some embodiments, the MCI protein comprises an amino acid sequence having at least 95% identity to a conserved domain of Xenopus or mammalian MCI, e.g., the coiled coil or TIRT domain, and retains at least one MCI activity. In some embodiments, the MCI protein comprises an amino acid sequence having at least 85, 90, 91, 92, 93, 94, 95, 96, 97, 98, 99, or 100% identity to sequence selected from the group consisting of: amino acids 178-215 of SEQ ID NO:2; amino acids 161-227 of SEQ ID NO:2; amino acids 178-374 of SEQ ID NO:2; SEQ ID NO:2; amino acids 172-238 of SEQ ID NO:6; amino acids 172-379 of SEQ ID NO:6; SEQ ID NO:6; amino acids 176-242 of SEQ ID NO:7; and SEQ ID NO:7. In some embodiments the MCI protein is selected from the group consisting of: amino acids 178-215 of SEQ ID NO:2; amino acids 161-227 of SEQ ID NO:2; amino acids 178-374 of SEQ ID NO:2; SEQ ID NO:2; amino acids 172-238 of SEQ ID NO:6; amino acids 172-379 of SEQ ID NO:6; SEQ ID NO:6; amino acids 176-242 of SEQ ID NO:7; and SEQ ID NO:7. In some embodiments, the MCI nucleic acid comprises a nucleic acid sequence having at least 85, 90, 91, 92, 93, 94, 95, 96, 97, 98, 99, or 100% identity to SEQ ID NO:1. Further provided are isolated MCI proteins as described above.

Further provided are expression vectors, e.g., viral vectors, comprising the MCI nucleic acid. Further provided are transgenic (recombinant) cells expressing MCI, e.g., recombinant MCI. In some embodiments, the cell is a ciliate cell, e.g., a multiciliate cell. In some embodiments, the cell is an epithelial cell, e.g., from a mucosal tissue.

In some embodiments, antibodies that specifically recognize an MCI protein, as described above, are provided, i.e., MCI antibodies, anti-MCI antibodies, or MCI-specific antibodies. In some embodiments, the MCI-specific antibody recognizes more than one species homolog of MCI, e.g., mouse and human MCI. In some embodiments, the MCI-specific antibody recognizes Xenopus MCI, mouse MCI, and/or human MCI. In some embodiments, the MCI-specific antibody is homolog-specific. In some embodiments, the MCI-specific antibody is specific for a conserved epitope on the MCI protein. In some embodiments, the MCI-specific antibody is an antagonist antibody. In some embodiments, the MCI-specific antibody is attached (directly or indirectly) to a detectable moiety.

Further provided are methods for preparing (forming, generating, making) a recombinant, MCI-expressing cell, comprising (i) introducing (e.g., transfecting) a nucleic acid encoding an MCI protein as described herein to a cell and (ii) allowing expression of said MCI protein in the cell, thereby preparing a recombinant, MCI-expressing cell. In some embodiments, the method comprises increasing the expression or activity of an endogenous MCI protein in a cell, thereby preparing a recombinant, MCI-expressing cell. In some embodiments, the MCI-expressing cell is a ciliate cell, e.g., a multiciliate cell. In some embodiments, the nucleic acid is introduced into a non-ciliate cell, and the MCI expression induces formation of cilia on the cell. In some embodiments, the MCI-expressing cell divides at least once before forming a ciliate cell.

Further provided are methods of diagnosing a ciliate cell related disorder or risk of developing a ciliate cell related disorder in an individual, said method comprising (i) determining the expression level of a multicilin (MCI) gene in a sample from the individual; and (ii) comparing the expression level from step (i) to the expression level of an MCI gene in a standard control sample, wherein a decreased MCI expression level relative to the standard control sample indicates that the individual has a ciliate cell related disorder. In some embodiments, the ciliate cell related disorder is selected from the group consisting of asthma, cystic fibrosis, mucus overproduction, primary ciliary dyskinesia (PCD), Kartegener's syndrome, nephronophthisis (Senior-Loken syndrome), respiratory tract infection, otitis, deficiency in Fallopian tube cilia, bronchiectasis, and hydrocephaly.

In some embodiments, where decreased MCI expression is determined, the method of diagnosis can further comprise administering to said individual an MCI agonist, e.g., a vector encoding an MCI protein. In some embodiments, the MCI agonist is administered to the respiratory tract, e.g., via an aerosol, nebulizer, or inhalant. In some embodiments, the MCI agonist is administered to the reproductive tract, e.g., using a cream or suppository.

For methods of diagnosis, said determining can comprise detection of MCI mRNA, e.g., using PCR or a hybridization based assay such as a Northern blot or array. In some embodiments, said determining comprises detecting MCI protein (e.g., using an MCI-specific antibody). In some embodiments, said determining comprises detecting MCI activity, e.g., production of multiciliate cells. In some embodiments, the diagnostic methods are carried out in vitro, e.g., using a sample obtained from the individual, e.g., an epithelial tissue sample. The tissue sample can be obtained using methods known in the art, e.g., from lung, ear, respiratory tract or airway tissue, brain (e.g., ventricular lining), or a reproductive tract sample. In some embodiments, the diagnostic methods are carried out in vivo, e.g., using imaging technology.

Further provided are methods of treating a ciliate cell related disorder in an individual, comprising administering an MCI agonist to the individual, thereby treating the ciliate cell related disorder. In some embodiments, the method of treatment comprises (i) determining the expression level of a multicilin (MCI) gene in a sample from the individual; (ii) comparing the expression level from step (i) to the expression level of an MCI gene in a standard control sample, wherein a decreased MCI expression level relative to the standard control sample indicates that the individual has a ciliate cell related disorder; and (iii) administering to an individual with decreased MCI expression an MCI agonist. In some embodiments, the ciliate cell related disorder is selected from the group consisting of asthma, cystic fibrosis, mucus overproduction, primary ciliary dyskinesia (PCD), Kartegener's syndrome, nephronophthisis (Senior-Loken syndrome), respiratory tract infection, otitis, deficiency in Fallopian tube cilia, bronchiectasis, and hydrocephaly.

In some embodiments, the MCI agonist is a vector encoding an MCI protein as described herein. In some embodiments, the vector is a viral vector. In some embodiments, the vector is a non-viral vector, e.g., administered using a liposomal or other lipid delivery vehicle. In some embodiments, the MCI agonist is administered to the respiratory tract, e.g., via an aerosol, nebulizer, or inhalant. In some embodiments, the MCI agonist is administered to the reproductive tract, e.g., using a cream or suppository.

### BRIEF DESCRIPTION OF THE DRAWINGS

FIG. 1: Schematic depicting regulation of ciliate cell differentiation.

FIG. 2: Structure of MCI and MCI deletion mutants.

FIG. 3: Multiciliate cells are marked by alpha-tubulin expression in the skin (A) and at later stages in the kidney (B, arrow). MCI expression occurs early in the developing skin (C, NP=neural plate) in a spotty pattern, disappears by stage 16, but reappears in the nephrostomes in the kidney (D, arrow). Insets in panels C and D show high magnification images.

FIG. 4: Embryos injected with the control construct, the MCI splice morpholino, or MCI-encoding sequence as indicated, and stained with ZO-1 and acetylated tubulin antibodies to detect cell boundaries and cilia, respectively. Asterisks denote unusually large cells in C. Bar=20 microns.

FIG. 5: RNAs encoding MCI-HGR (top right) or two deletion mutants (bottom panels) were injected into embryos that were fixed and stained as in FIG. 4. Confocal images. Bar=20 microns. Arrowhead denotes ciliated cell, and arrow denotes PSCs (Pseudostratified Columnar cells).

FIG. 6: (A) Outer layer cells (OC) and inner layer cells have different fates, giving rise to (B) mucus-secreting cells (goblet cells) or to PSCs and ciliated cells (CCs), respectively.

FIG. 7: Deletion of coiled coil region of MCI blocks cilia formation in cells.

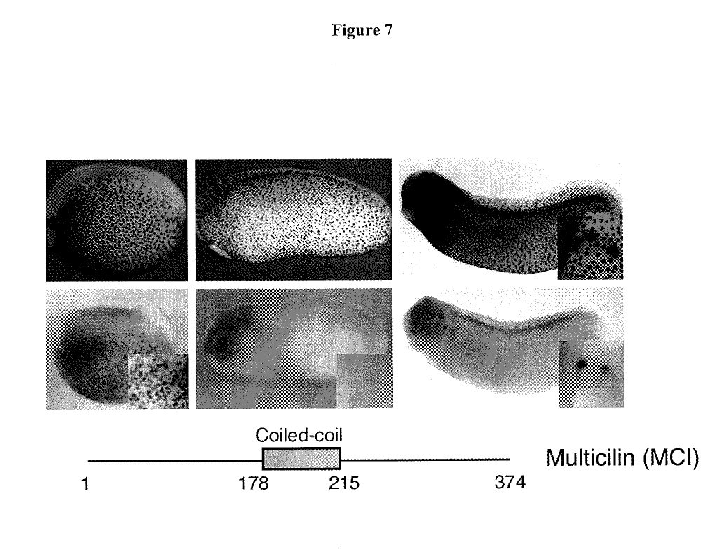

FIG. 8: MCI morpholino blocks the appearance of multiciliate cell precursors.

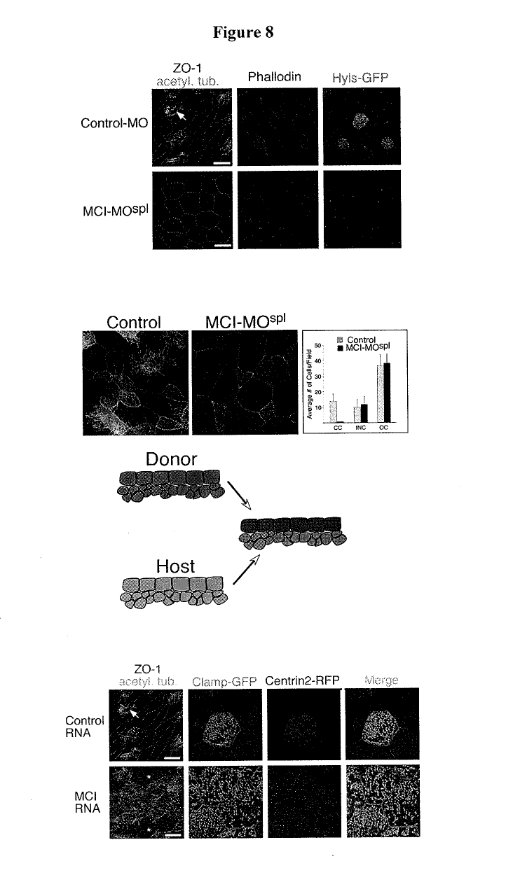

FIG. 9: MCI induces multiciliate cells in both layers of the ectoderm.

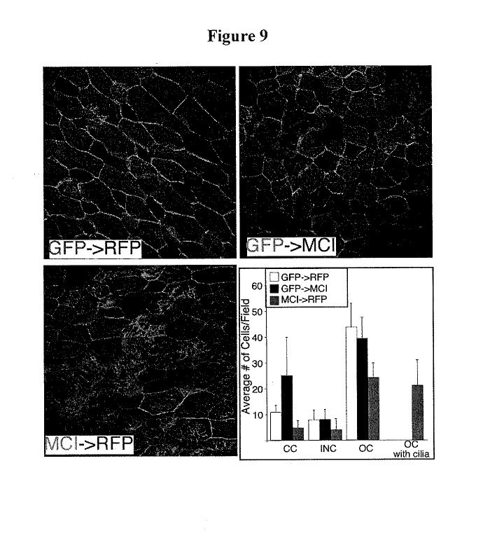

FIG. 10: MCI is involved in multiciliate cell differentiation.

FIG. 11: Amino acid sequence alignment of Xenopus (bc124892; SEQ ID NO:2) and mouse MCI (mMCI1; SEQ ID NO:6). The “Majority” consensus sequence is SEQ ID NO:4.

FIG. 12: Amino acid sequence alignment of Xenopus (Xl), mouse (Mu) and human (H) MCI. The “Majority” consensus sequence is SEQ ID NO:5. The Xenopus sequence shown represents amino acids 1-295 of SEQ ID NO:2. The mouse sequence represents amino acids 1-303 of SEQ ID NO:6. The human MCI protein sequence is shown as SEQ ID NO:7.

FIG. 13: Multicilin antibody is specific for MCI: Xenopus embryos were injected with mRNA encoding Flag or Myc tagged MCI, Myc tagged GEMC1 (geminin coiled-coil containing protein 1, a related protein) or were uninjected. At stage 9 animal caps were removed and homogenized. Homogenates were then run out on a 10% SDS-PAGE gel. Blots with the anti-MCI antibody show specific bands at expected sizes for Flag and Myc tagged MCI but not GEMC1 (see lanes 2, 3 and 4). The anti-myc tag blot shows that both Myc-MCI and Myc-GEMC1 are expressed (lanes 7, 8).

FIG. 14: MCI is expressed in ciliated cells in mouse trachea: Mouse tracheal epithelial cell (MTEC) cultures were induced to differentiate into multiciliate cells by placing cultures at air-liquid interface (ALI). Within 2 hours (ALI+0d) cultures express MCI and continue to express MCI through the first 2 days of differentiation (ALI+2d). Tracheal cells from transgenic FoxJ1/EGFP mice were isolated and FACS sorted. Only multiciliate cells express EGFP under control of the FoxJ1 promoter and only EGFP+ cells express MCI (compare lanes 9 and 11). Controls with no DNA do not express MCI or the control gene GAPDH.

FIG. 15: Expression of Xenopus or mouse MCI induces multiciliate cells: Xenopus embryos were injected with RNA encoding membrane RFP (mRFP) to mark the injected region along with Xenopus (B) or mouse (C) MCI. In embryos injected with mRFP alone (A) ciliated cells are evenly spaced (green staining). Upon injection of either Xenopus (B) or mouse (C) MCI almost all cells in the skin are ciliated.

### DETAILED DESCRIPTION OF THE INVENTION

### I. Introduction

Provided herein is are methods and compositions relating to cilia formation that can be used for diagnosis and treatment of conditions involving cilated cells. The inventors have characterized the multicilin (MCI) protein and discovered that it is necessary and sufficient for formation of multicilate cells. Considering the primary role that ciliated cells play in proper maintenance of mucosal tissues, e.g., in the airway, nasal passages, brain and reproductive system, recognition of a crucial ciliate cell protein represents an important advance in the field.

### II. Definitions

Multicilin (MCI) is a protein encoded by the MCI gene that is transiently expressed in ciliated cell development. MCI is involved in differentiation and cell cycle regulation that results in formation of cilia. The gene or protein can be naturally occurring (e.g., a wild type or polymorphic form, or naturally occurring mutant found in an organism) or recombinant, synthetic, or otherwise manipulated. Thus, for example, a human MCI protein is a protein having a sequence found in a human.

Cilia are slender projections from a cell that can be either motile or non-motile. The structure is provided by a microtubule based axoneme. At the base of the cilium is a basal body. Motile cilia are usually present on a cell surface in large numbers and beat in coordinated waves. Cells with motile cilia are thus usually multiciliate cells, e.g., ciliated epithelial cells, which are found in mucosal epithelia in the respiratory and reproductive systems and in the brain. Many cell types have a non-motile cilium (e.g., a primary cilium) (Gardiner (2005) HHMI Bulletin 18).

A ciliate cell disorder or ciliate cell related disorder is a condition caused or exacerbated by dysregulation of ciliated cells. Ciliopathies are a subset of these disorders, which find their underlying cause in cilia dysfunction. Problems typically arise because of reduced ciliary function, e.g., where cilia are not formed normally, are produced at reduced levels, or do not function sufficiently. In some cases, cilia are over-produced or over-active, which can lead to increased mucosal flow or mucosal deficiency. Ciliate cell disorders are reviewed, e.g., in Afzelius (2004) J. Pathol. 204:470 and Badano et al. (2006) Ann. Rev. Genomics and Hum. Genet. 7:125.

The term “isolated,” when referring to a cell or molecule (e.g., nucleic acid or protein), indicates that the cell or molecule is or has been separated from its natural environment. For example, an isolated cell can be removed from its host individual, but still exist in culture with other cells, or be reintroduced into its host individual.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The terms “identical” or “percent identity,” in the context of two or more nucleic acids, or two or more proteins, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 15, 20, 25, 30, 35, 40, 45 or more amino acids or nucleotides in length, e.g., 25-40, 30-40, 30-45, 40-80, or over a region that is 50-100 amino acids or nucleotides in length. In some cases, the percent identity of corresponding (e.g., homologous) sequences is determined over the length of a functional domain, more than one functional domain, or over the entire length of the sequence. The percent identity of corresponding functional domains (and sequences encoding functional domains) is typically higher than that of intervening sequences.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid can be used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The term “gene” refers to a segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Gene products include transcripts of the gene that typically do not include the regulatory sequences or introns (e.g., mRNA) and protein. A “protein gene product” is a protein expressed from a particular gene and encoded by the transcript. The word “expression” or “expressed” as used herein in reference to a gene means the transcriptional and/or translational product of that gene.

A “vector” is a nucleic acid that is capable of transporting another nucleic acid into a cell. A vector is capable of directing expression of a protein or proteins encoded by one or more genes carried by the vector when it is present in the appropriate environment.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The antisense constructs “short hairpin RNA” and “small hairpin RNA” are ribonucleotide sequences forming a hairpin turn which can be used to silence gene expression. After processing by cellular factors the short hairpin RNA interacts with a complementary RNA thereby interfering with the expression of the complementary RNA.

The words “protein,” “peptide,” and “polypeptide” refer an amino acid polymer or a set of two or more interacting or bound amino acid polymers. The terms can also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or substantially identical amino acid sequences, or where the nucleic acid does not encode an amino acid sequence, to substantially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Substitution of a given residue with a different amino acid that does not result in a substantial change in the activity of the protein can indicate that the substitution is conservative.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “dominant negative protein” is a modified form of a wild-type protein that adversely affects the function of the wild-type protein within the same cell. As a modified version of a wild-type protein the dominant negative protein may carry a mutation, a deletion, an insertion, a post-translational modification or combinations thereof. Any additional modifications of a nucleotide or polypeptide sequence known in the art are included. The dominant-negative protein may interact with the same cellular elements as the wild-type protein thereby blocking some or all aspects of its function.

“Antibody” refers to a polypeptide comprising a framework region from an immunoglobulin gene or fragments thereof that specifically bind to and recognize an antigen. Antibodies bind to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

A “label” or a “detectable moiety” is a composition detectable by spectroscopic, photochemical, biochemical, immunochemical, chemical, or other physical means. For example, useful labels include fluorescent dyes, ³²P and other radionucleotides, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and animals are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

A “control” or “standard control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known ciliated cell or a cell exposed to known conditions or agents, for the sake of comparison to the test condition. A control can also represent an average value gathered from a plurality of samples, e.g., to obtain an average value. A control or standard control can also represent a sample to ensure that the assay is functional, e.g., a housekeeping gene as a standard control in an expression assay.

For therapeutic applications, a sample obtained from a patient suspected of having a given disorder or deficiency can be compared to samples from a known normal (non-diseased or non-affected) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “agonist,” “activator,” “upregulator,” etc. refer to a substance capable of detectably increasing the expression or activity of a given gene or activity. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or more higher than the expression or activity in the absence of the agonist.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

“Subject,” “patient,” and like terms are used interchangeably and refer to animals, typically mammals such as humans and non-human primates, as well as rabbits, rats, mice, goats, pigs, and other mammalian species. The term does not necessarily indicate that the subject has been diagnosed with a particular disease, but typically refers to an individual under medical supervision.

The term “diagnosis” refers to a relative probability that a ciliate cell disorder is present in the subject. Similarly, the term “prognosis” refers to a relative probability that a certain future outcome may occur in the subject. For example, in the context of the present invention, prognosis can refer to the likelihood that an individual will develop a disease involving a mucosal tissue such as the airway, or the likely severity of the disease (e.g., severity of symptoms, rate of functional decline, survival, etc.). The terms are not intended to be absolute, as will be appreciated by any one of skill in the field of medical diagnostics.

The terms “correlating” and “associated,” in reference to determination of a ciliate cell disorder risk factor, refers to comparing the presence or amount of the risk factor (e.g., dysregulation or genetic variation in a mucin gene) in an individual to its presence or amount in persons known to suffer from, or known to be at risk of, the ciliate cell disorder, or in persons known to be free of a ciliate cell disorder, and assigning an increased or decreased probability of having/developing the disorder to an individual based on the assay result(s).

As used herein, the terms “treat” and “prevent” are not intended to be absolute terms. Treatment can refer to any reduction in the frequency or severity of symptoms, amelioration of symptoms, improvement in patient comfort and/or function, etc. The effect of treatment can be compared to an individual or pool of individuals not receiving a given treatment, or to the same patient prior to, or after cessation of treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The term “prevent” refers to a decrease in the occurrence of symptoms of a ciliate cell disorder in a patient. As indicated above, the prevention may be complete (no detectable symptoms) or partial, such that fewer symptoms are observed than would likely occur absent treatment.

The term “therapeutically effective amount,” as used herein, refers to that amount of the therapeutic agent sufficient to ameliorate the disorder. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

### III. Recombinant Methods

Technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989).

The term “transfection” or “transfecting” is defined as a process of introducing a nucleic acid molecule to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, direct injection, and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art.

Expression of a transfected gene can occur transiently or stably in a host cell. During “transient expression” the transfected nucleic acid is not integrated into the host cell genome, and is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision.

The term “transduction” as used herein refers to introducing protein into a cell from the external environment. Typically, transduction relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A variety of methods of specific DNA and RNA measurements that use nucleic acid hybridization techniques are known to those of skill in the art (see, e.g., Sambrook). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

In some aspects, amplification of known sequences may be desirable, e.g., for cloning into appropriate expression vectors, increasing the sensitivity of hybridization-based assays, or direct detection. Such methods of amplification are well known to those of skill in the art. Detailed protocols for PCR are provided, e.g., in Innis et al. (1990) PCR Protocols, A Guide to Methods and Applications, Academic Press, Inc. N.Y.). The known nucleic acid sequences for the genes listed herein is sufficient to enable one of skill to routinely select primers to amplify any portion of the gene.

Examples of such systems include the polymerase chain reaction (PCR) system and the ligase chain reaction (LCR) system. Other methods include the nucleic acid sequence based amplification (NASBA, Cangene, Mississauga, Ontario) and Q Beta Replicase® systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products in real time.

In some aspects, a nucleotide sequence that increases expression of a protein of the invention can be used, e.g., an MCI-encoding sequence. In some embodiments, a promoter, e.g., an inducible promoter, can be placed proximal to the MCI-coding sequence such that it is operably attached and controls MCI expression. In some embodiments, a nucleic acid that specifically interferes with expression of MCI at the transcriptional or translational level can be used. This approach may utilize, for example, siRNA and/or antisense oligonucleotides to block transcription or translation of a specific mRNA, either by inducing degradation of the mRNA with a siRNA or by masking the mRNA with an antisense nucleic acid.

The siRNA is typically about 5 to about 100 nucleotides in length, more typically about 10 to about 50 nucleotides in length, most typically about 15 to about 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

Antisense nucleic acids are DNA or RNA molecules that are complementary to at least a portion of a specific mRNA molecule (see, e.g., Weintraub, Scientific American, 262:40 (1990)). Typically, synthetic antisense oligonucleotides are generally between 15 and 25 bases in length. Antisense nucleic acids may comprise naturally occurring nucleotides or modified nucleotides such as, e.g., phosphorothioate, methylphosphonate, and -anomeric sugar-phosphate, backbone-modified nucleotides.

### IV. Ciliated Cells and Related Disorders

Ciliated cells can be found in the epithelia of the respiratory tract and reproductive tract (e.g., Fallopian tubes), in the kidney, brain (e.g., ventricular ependymal cells). Ciliated cells also play a role during development, where nodes in gastrulation determine symmetry.

Motile cilia propel fluid across the surface of an epithelial sheet. Cilia-driven flow is strongly directional and extends over long distances relative to a cell. Ciliated cells play a role in proper maintenance of tissues in the airway, brain, reproductive organs, etc. Disordered cilia formation or activity can result in improper clearance of mucus and associated pollutants, allergens, and pathogens, etc. Disorders related to ciliate cells can be divided into those that result from reduced production or activity of ciliated cells, e.g., non-effective or aberrant ciliate cell activity, and those that result from over-production or excess activity of ciliated cells. The majority of disorders that are recognized as being related to ciliated cells are in the first category.

Reduced ciliary activity in the airway can result in primary ciliary dyskinesia (PCD), blocked mucus transport, bronchiectasis and chronic sinusitis. Loss of ciliary flow in the female reproductive tract can misplace the egg, causing ectopic pregnancies. In the brain, ciliated ependymal cells produce long-range flow of cerebrospinal fluid (CSF) within the ventricles. This flow directs the long range migration of neuroblasts along the lateral walls to the olfactory bulb, and defects can result in pressure build-up and hydrocephalus.

Examples of ciliate cell related disorders include asthma, cystic fibrosis, mucus overproduction, primary ciliary dyskinesia (PCD), Kartegener's syndrome, nephronophthisis (Senior-Loken syndrome), respiratory tract infection, respiratory inflammation (e.g., due to insufficient clearance of allergens or pollutants), otitis, deficiency in Fallopian tube cilia, bronchiectasis, and hydrocephaly.

Over-production of cilia or ciliated cells can result in over-clearance or aberrant flow of fluids such mucus. In the female reproductive tract, insufficient mucus can interfere with fertilization. In the airway, insufficient mucus can result in irritation and irregular clearance of pathogens, pollutants and allergens.

Ciliate cell disorders are reviewed, e.g., in Afzelius (2004) J. Pathol. 204:470 and Badano et al. (2006) Ann. Rev. Genomics and Hum. Genet. 7:125.

### V. Multicilin

Multicilin is a coiled-coil protein that is involved in cell cycle regulation and development of ciliated cells, e.g., multiciliate cells. Multicilin (MCI) expression can be detected by detecting MCI transcripts (mRNA), protein, or MCI activity. For example, determining the number of multiciliated cells in a tissue sample, relative to a standard control, can be indicative of MCI activity. Similarly, determining the size of a ciliate cell or ciliate cell precursor can be indicative of MCI cell cycle activity.

MCI can be detected using standard methods of RNA or protein detection. Aberrant expression of MCI can be associated with a ciliate cell disorder, or likelihood of developing a ciliate cell disorder. For example, reduced MCI expression can result in reduced production of multiciliate cells and reduced mucus flow. A modulated RNA level, protein level, or other measured quantity relative to a control standard is one that differs from the measured or calculated amount of the same quantity in the standard control. In some embodiments, the measured quantity is greater than or elevated above or increased over the standard control greater by or elevated above or increased over the standard control by at least 5%, 10%, 15%, 20%, 25%, 30%, 35%, 40%, 50%, 60%, 75%, 80%, 90%, or at least 100%. In certain embodiments, the measured quantity is less than or decreased compared to the standard control. In other embodiments, the measured quantity is less than or decreased as compared to the standard control by at least 5%, 10%, 15%, 20%, 25%, 30%, 35%, 40%, 50%, 60%, 75%, 80%, 90%, or 100%. The modulated level or amount of a measured quantity (e.g. MCI RNA or protein) can be also be expressed as a “-fold” increase or decrease. For example, the measured quantity can be at least 1.1-fold, 1.2-fold, 1.5-fold, 2-fold, 5-fold, or larger-fold less than a standard control.

Methods for detecting and identifying nucleic acids and proteins involve conventional molecular biology, microbiology, and recombinant DNA techniques within the skill of the art. Such techniques are explained fully in the literature (see, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

For detection, a detectable moiety can be associated with an antibody, primer or probe of the invention, either directly, or indirectly, e.g., via a chelator or linker, to form a detection agent, e.g., for detecting MCI or another ciliated cell marker. The detection agent can then be used in an in vitro assay that includes a biological sample from an individual, or can be provided to the individual to determine the applicability of an intended therapy. For example, a labeled antibody may be used to detect the density of multiciliate cells within a diseased area. Administration of a detection agent can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.).

A detection agent (or diagnostic agent) for MCI or another ciliate cell marker can include any detection agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The terms “detectable agent,” “detectable moiety,” “label,” “imaging agent,” and like terms are used synonymously herein.

A radioisotope can be incorporated into the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In some embodiments, the detection agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the detection agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

### VI. MCI-Specific Antibodies

Antibodies of the invention include those that can specifically bind to (recognize, detect) MCI proteins. In some embodiments, the antibody detects MCI from more than one species, e.g., mouse, Xenopus, and/or human. For example, such antibody can be specific for an epitope on a relatively well-conserved region or surface of the protein (e.g., the coiled coil or the TIRT region). In some embodiments, the antibody is specific for the MCI protein from a single species. In some embodiments, the antibody is specific for a particular form of the MCI protein, e.g., a post-translationally modified form. In some embodiments, the antibody interferes with MCI activities, e.g., an antagonist antibody.

The antibody can be generated in any mammal. Antibodies against human proteins are often initially raised in rodent or rabbit immunoglobulin producing cells, and can be further manipulated as described below. The antibody can be monoclonal or polyclonal.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein. Such effector moieties include, but are not limited to, a mucus-reducing drug, a toxin, a radioactive agent, a cytokine, a second antibody or an enzyme.

The immunoconjugate can be used for targeting the effector moiety to a ciliated cell. Suitable detectable markers include, but are not limited to, a radioisotope, a fluorescent compound, a bioluminescent compound, chemiluminescent compound, a metal chelator or an enzyme.

Techniques for conjugating detectable and therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

### VII. Modulators of MCI

The invention further provides methods for identifying antagonists or agonists of MCI expression and/or activity. Methods for screening for antagonists or agonists may include measuring the ability of the potential antagonists or agonist to reduce or increase an identifiable MCI activity or compete for binding with a known binding agent (e.g., an MCI-specific antibody). For example, candidate agents can be screened for their ability to increase an MCI activity or expression. An exemplary agonist is a vector comprising an MCI-encoding nucleic acid. Some embodiments include identifying antagonists of MCI. Exemplary antagonists include a morpholino (antisense sequence) specific for the sequence of MCI and an antagonist MCI-specific antibody.

The screening methods of the invention can be performed as in vitro or cell-based assays or in suitable animal models (e.g. in a cilia-deficient mouse model). Thus, in some embodiments, the method can include contacting MCI, an MCI-expressing cell, or a ciliate cell with a candidate agent. The cell, in turn, may form part of a tissue and/or an organism (i.e. an animal model). Cell based assays can be performed in any cells in which MCI is expressed or not expressed, either endogenously or through recombinant methods. Suitable cell-based assays are described in, e.g., DePaola et al., Annals of Biomedical Engineering 29: 1-9 (2001).

Agents that are initially identified as modulating MCI can be further tested to validate the apparent activity. Preferably such studies are conducted with suitable cell-based or animal models of disease (e.g. ciliate cell disorders). The basic format of such methods involves administering a lead compound identified during an initial screen to an animal that serves as a model and then determining if in fact the disease or one or more of the disease symptoms are ameliorated. The animal models utilized in validation studies generally are mammals of any kind. Specific examples of suitable animals include, but are not limited to, primates (e.g., chimpanzees, monkeys, and the like) and rodents (e.g., mice, rats, guinea pigs, rabbits, and the like).

The agents tested as potential agonists or antagonists of MCI can be any appropriate small chemical compound, or a biological entity, such as a polypeptide, sugar, nucleic acid or lipid. Alternatively, modulators (e.g. agonists or antagonists) can be genetically altered versions of MCI, e.g., dominant negative or constitutively active forms. Essentially any appropriate chemical compound can be used as a potential modulator in the assays of the invention. The assays can be designed to screen large chemical libraries by automating the assay steps and providing compounds from any convenient source to assays, which are typically run in parallel (e.g., in microtiter formats on microtiter plates in robotic assays).

In one embodiment, high throughput screening methods are employed by providing a combinatorial chemical or peptide library containing a large number of candidate MCI binding agents. Such “combinatorial chemical libraries” or “ligand libraries” are then screened in one or more assays, to identify those library members (particular chemical species or subclasses) that display a desired characteristic activity. The candidate compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, and U.S. Pat. No. 5,288,514).

### VIII. Therapeutic Methods and Compositions

MCI provides both a therapeutic and diagnostic target for ciliate cell related disorders. Accordingly, provided herein are methods of determining whether an individual is at risk of developing a ciliate cell disorder, or indeed suffers from a ciliate cell disorder, comprising detecting (determining) the level of MCI expression and comparing it to a standard control, wherein a modulated or aberrant MCI expression level relative to the control is indicative that the individual is at risk of developing or has a ciliate cell disorder. In addition, provided herein are methods of treating an individual with a ciliate cell disorder by administering an MCI agonist or antagonist to the individual, depending on the particular ciliate cell disorder to be treated.

The method of determining or method of treating can include obtaining a sample from the subject or individual. The sample can be a tissue sample, e.g., an epithelial cell sample. In some embodiments, the method of determining or method of treating includes isolating cells from the epithelia, thereby forming an isolated cell sample. In some embodiments, the method of determining or method of treating includes obtaining cells, e.g., from a bronchioaviolar lavage sample, or other mucosal lining sample.

Provided herein are methods and compositions for regulating MCI expression in appropriate tissues. For example, aberrant over-expression of MCI in a tissue that is normally lacking MCI expression can be addressed by administration of an antisense sequence specific for MCI or an MCI-specific antagonist antibody.

More commonly, increased MCI expression (e.g., inducible expression) is desired. MCI agonists, e.g., nucleic acid vectors that comprise MCI-encoding sequences can be administered to a mucosal tissue using known methods. The therapeutic and diagnostic compositions of the invention include viral vectors, e.g., adenoviral, adeno-associated, or lentiviral constructs. Methods and compositions for delivering viral vectors to respiratory tissues are described, e.g., in Sinn et al. (2005) Am J Respiratory Cell Mol. Biol. 32:404, which describes use of viscoelastic gel formulations to aid delivery. Non-viral delivery vectors for airway gene delivery are described, e.g., in Davis and Cooper (2007) AAPS 9:E11-E17. Viral and non-viral delivery systems for airway and other mucosal tissues are further described in Duan et al. (2001) Curr. Prot. Hum. Genet. Unit 13.9.

Therapeutic and diagnostic compositions for administration typically include a pharmaceutically acceptable excipient, which term is used herein according to its generally recognized meaning in the pharmaceutical arts. It may refer to any substance included in a pharmaceutical composition, which is generally considered to be not an active ingredient, that is commonly employed in order to improve administration or absorption of the active ingredient. In some embodiments, excipients facilitate the manufacture, administration, storage, or efficacy of an active ingredient. Examples of excipients include antiadherents, binders, coatings, disintegrants, fillers, diluents, flavors, colors, lubricants, glidants, preservatives, sorbents, or sweeteners.

The compositions disclosed herein can be administered by any means known in the art. In some cases, compositions of the invention are injected (either bolus or infusion) or otherwise applied to the affected site. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, locally, by inhalation, by injection, by infusion, by continuous infusion, by localized perfusion, via a catheter, via a lavage, in a vaginal suppository cream or composition, suspension, or lipid composition. Administration can be local, e.g., to the affected tissue, or systemic.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines.

For parenteral administration in an aqueous solution, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by sterile filtration of the media or injection vehicle prior to incorporating the cells for injection. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more concentrated solutions for direct injection is also contemplated. DMSO can be used as solvent for rapid penetration, delivering high concentrations of the active agents to a small area.

In some embodiments, a plurality of MCI-expressing cells can be introduced to an appropriate tissue in an individual. In this case, the pharmaceutical compositions of the invention can optionally comprise growth factors or cell matrix components to support growth of differentiated endothelial, cilated cells. For example, the cells can be administered in a solution of matrigel, optionally comprising, e.g., cell growth factors.

The invention provides methods of treating, preventing, and/or ameliorating a ciliate cell disorder in a subject in need thereof. The course of treatment is best determined on an individual basis depending on the particular characteristics of the subject and the type of treatment selected. The therapeutic compositions of the inventions can be administered to the subject on a weekly, monthly, or any applicable basis that is therapeutically effective. The treatment can be administered alone or in combination with any other treatment appropriate for a ciliate cell disorder, or for improving patient comfort, e.g., to reduce mucus secretion or pain or swelling. The additional treatment can be administered simultaneously with the first treatment, at a different time, or on an entirely different therapeutic schedule (e.g., the first treatment can be daily, while the additional treatment is weekly).

### IX. EXAMPLES

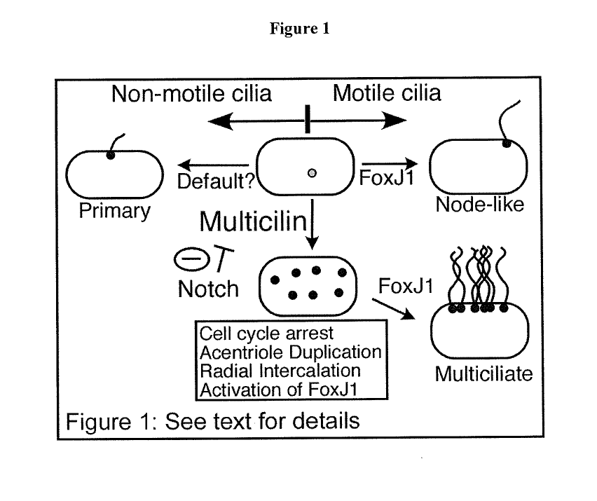

We found that Notch signaling negatively regulates the formation of multiciliate cells in the X. laevis larval skin in a manner consistent with the process of lateral inhibition (Deblandre et al. (1999) Development 126:4715-28). As multiciliate cell precursors arise in the skin, they express a Notch ligand, activate the Notch receptor on neighboring cells, and suppress multiciliate cell differentiation. Accordingly, activating Notch in the skin constitutively blocks the formation of multiciliate cell precursors and FoxJ1 expression is lost. Inhibiting Notch results in a marked increase in multiciliate cell precursors, and FoxJ1 expression is expanded (FIG. 1). If FoxJ1 expression is induced by RNA injection, and Notch is also activated, multiciliate cell differentiation is not rescued, only the ability to form a motile cilium (Stubbs et al. (2008) Nat Genetics 40:1445). Notch likely represses aspects of early multiciliate cell differentiation. This model not only applies to the X. laevis skin, but also holds true for other tissues where multiciliate cells form (see e.g. Guseh et al. (2009) Development 136:1751; Morimoto et al. (2010) J Cell Sci 123:213-14; Tsao et al. (2009) Development 136: 2297; Liu et al. (2007) Development 134:1111; Ma et al. (2007) PLoS Genet 3:18).

### Example 1

### Identification of Multicilin

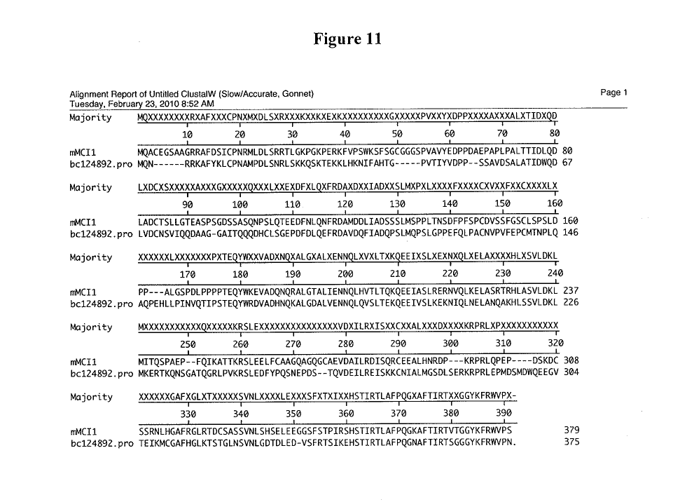

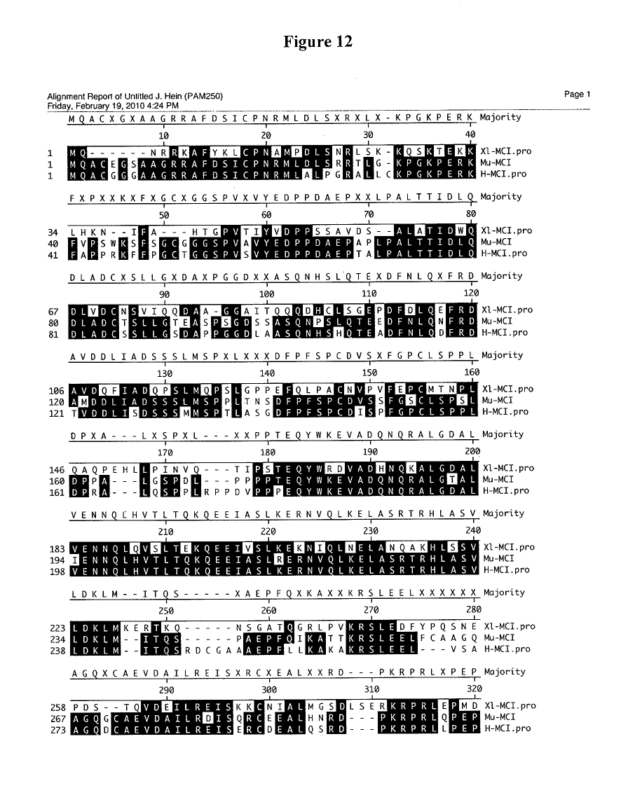

Based on studies of Notch signaling and multiciliate cell formation, we proposed the model outlined in FIG. 1. To test this model, we searched for genes in X. laevis that underlie the formation of multiciliate cells based on lineage-restricted expression and Notch signaling. Accordingly, embryos were injected with RNAs encoding agents that activate or repress Notch signaling. Total RNA was isolated from stage 12 skin, when multiciliate cell precursors first arise, and gene profiling was carried out by hybridization to the 2nd-generation X. laevis Affymetrix® gene arrays. The gene whose expression changed the most in this comparison corresponds to an unannotated X. tropicalis gene that we have called Multicilin (MCI). We have also found the mouse and human homologs of MCI, as shown in SEQ ID NOs:6 and 7 (see FIGS. 11 and 12).

MCI expression is highly restricted to tissues where multiciliate cells form. MCI is transiently expressed during multiciliate cell differentiation (see 1Bi below). Thus, MCI is expressed in a manner expected for a lineage-restricted determinant of multiciliate cell differentiation.

### Example 2

### Multicilin is Necessary and Sufficient for Multiciliate Differentiation

Injection of an embryo with an MCI antisense construct (a morpholino oligo, which blocks relatively short sequences (˜25 mers) of RNA), or a dominant-negative mutant, inhibits differentiation of multiciliate cells in the skin, perhaps as early as precursor formation (see 1Bii, below). Conversely, ectopic expression of MCI induces the formation of ectopic multiciliate cells in the skin, converting most, if not all, of the cells in the ectoderm into multiciliate cells (see 1Biii and 1Biv below). This phenotype is quantitatively and qualitatively different from simply disabling Notch signaling, and indeed still occurs in the presence of activated Notch, ICD. Thus, a single factor, MCI, is both necessary and sufficient to drive the formation of multiciliate cells. Finally, MCI acts in the nucleus to activate gene expression associated with early multiciliate cell differentiation, including the expression of FoxJ1. MCI is thus a key element in the transcriptional code required for the formation of this specialized epithelial cell type (FIG. 1)

### Example 3

### Multicilins Encode a Family of Novel Coiled-Coil Proteins

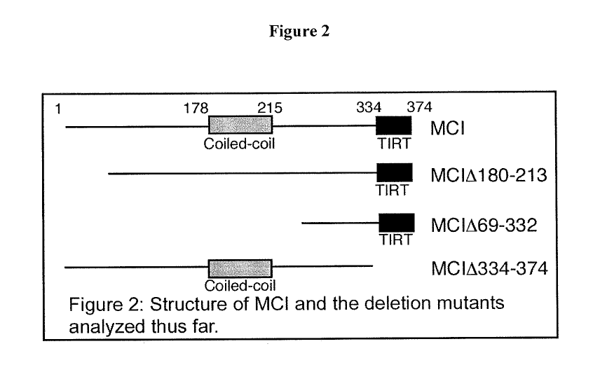

The structural features of the protein encoded by MCI are also consistent with its role as a regulator of multiciliate cell differentiation. The MCI proteins found in different vertebrate species are small, about ˜375aa in size, with a highly conserved central coiled-coil domain (FIG. 2). The N-terminal 100 amino acids are not well conserved, but those lying adjacent to the coiled-coil domain share significant blocks of homology unique to this family. The most striking homology outside the coiled-coil domain is a ˜30aa domain at the carboxy terminus, with over 90% amino acid identity among MCIs (see FIGS. 2, 11, and 12), hereafter referred to as the TIRT domain. The coiled-coil domain and the TIRT domain are involved in MCI function, based on the analysis of the deletion mutants (FIG. 2). The modular domains in MCI may mediate protein-protein interactions that lead to changes in transcriptional activity.

In addition to promoting multiciliate cell differentiation, MCI is a potent inhibitor of cell cycle progression (1Biii below). Analysis of the MCI, therefore, is required for multiciliate cell differentiation and proper cell cycle progression, and provides an important new genetic inroad into the formation of multiciliate cells, a unique cilia subtype.

Prior to the present disclosure, multiciliate cells formation and function in the context of epithelia in metazoans had been largely unexplored, outside of descriptive studies. One reason for this slow progress is a lack of suitable model systems: both C. elegans, and Drosophila, two of the classic genetic models for studying developmental mechanisms, do not have epithelia that project motile cilia and produce directed fluid flow. To address this deficiency, provided herein is a vertebrate model for the study of multiciliate cells, namely X. laevis, taking advantage of the fact that these cells form early on in the larval skin in a manner that is very accessible to experimental manipulation.

The X. laevis model provided herein can address basic mechanisms of multiciliate cell differentiation that can be applied to other vertebrates and tissues, such as the mammalian lung. Further provided herein is the discovery of Multicilin and its potential role a master regulator of multiciliate cell differentiation.

### Example 4

### Characterization of MCI Expression and Mutant Phenotypes

To determine whether MCI, a Notch-regulated gene in the developing X. laevis skin, plays a role in multiciliate cell differentiation, we examined the embryonic expression of MCI RNA. MCI function was tested in embryos in both gain- and loss-of-function experiments.

### 1Bi: Transient Expression of MCI During Multiciliate Cell Differentiation.

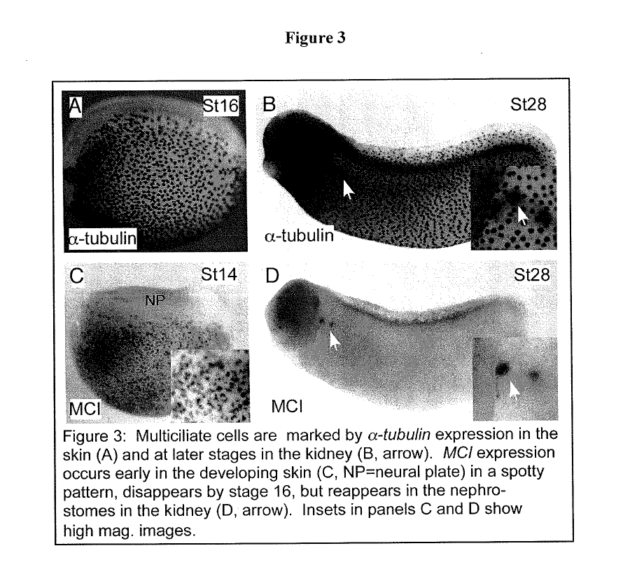

Whole mount in situ hybridization was used to localize the expression of MCI in X. laevis embryos. MCI expression is first detected during gastrulation, localizing to the ventral ectoderm where multiciliate cells form (FIG. 3C). Expression is spotty, suggesting lateral inhibition (FIG. 3C inset), and indeed, MCI expression is eliminated in embryos when Notch is activated, and expands to more cells when the Notch pathway is inhibited (according to methods disclosed, e.g., in Deblandre (1999) Development 126:4715). MCI expression in the skin is also transitory. Expression is lost by stage 26, when the multiciliate cells are functional and producing flow (FIG. 3).

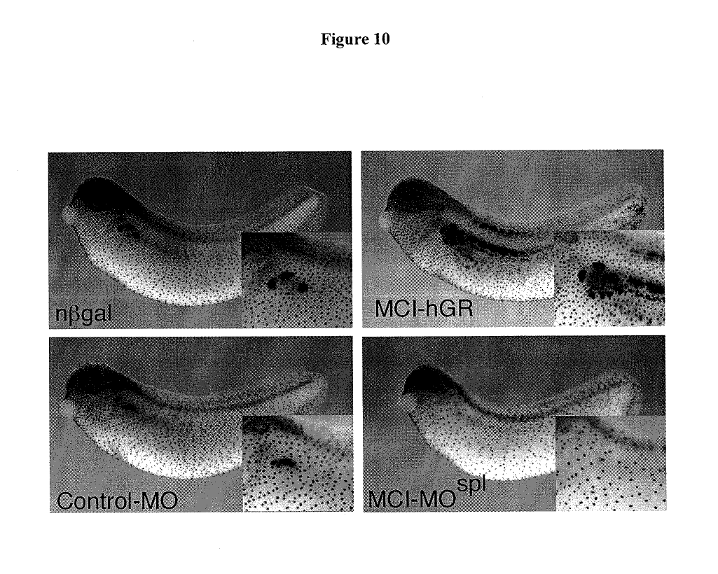

Yet MCI expression appears later in other tissues where multiciliate cells are known to form, notably, the developing nephrostomes of the kidneys (FIGS. 3D and 10), and areas surrounding the olfactory placodes (methods described in, e.g., Naylor et al. (2009) Development 136:3585). Thus MCI expression is transiently associated with multiciliate cell formation in several tissues.

### 1Bii: Knock Down of MCI Activity Inhibits the Formation of Multiciliate Cells in the Skin.

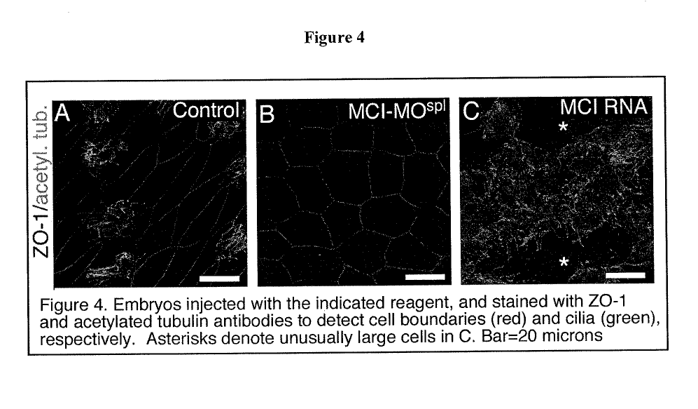

A loss-of-function (LOF) analysis of MCI activity was performed by designing morpholinos to target either the initiation ATG (MCI-MO^{ATG}) or a splice donor site lying at the junction between exon 4 and intron 5 (MCI-MO^{SPL}) (morpholino design described in Heasman (2002) Dev Biol. 243:209; Nasevicius & Ekker (2000) Nat Genet. 26:216). MCI-MO^{ATG} proved to be toxic to embryos, presumably as a consequence of off-site targets. MCI-MO^{SPL} morphants developed normally by external criteria, but failed to form multiciliate cells (FIG. 4B). In contrast to the phenotype in FoxJ1 morphants, the skin of MCI-MO^{SPL} morphants lacked cells with the characteristic morphology of multiciliate cells or with multiple basal bodies, based on Centrin-GFP localization (Stubbs et al. (2008) Nat Genet 40:1454). Thus, inhibiting MCI function results in a much earlier block in the differentiation of multiciliate cells than that observed in FoxJ1 mutants, perhaps as early as precursor formation.

### 1Biii: Ectopic Expression of MCI Induces Ectopic Multiciliate Cell Differentiation.

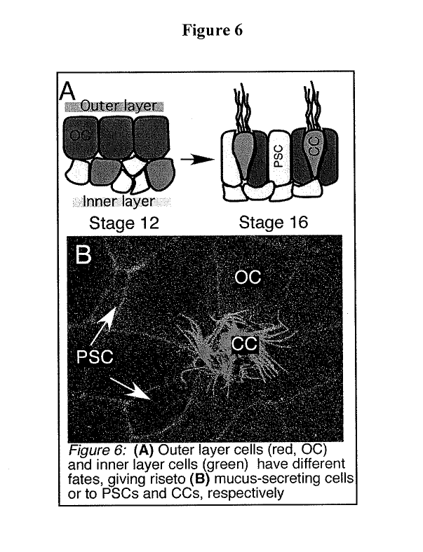

A gain-of-function (GOF) analysis of MCI was also performed, by injecting embryos with MCI RNA (FIG. 4C). Injection of a high concentration of MCI RNA proved toxic, resulting in large cells, and cell death at the start of gastrulation. At lower concentrations, MCI RNA injected embryos survived and showed two striking phenotypes. First, the cells comprising the outer epithelium were in some cases 2-4 larger in area than normal, indicating that MCI induces cell cycle arrest (asterisks in FIG. 4C). Second and more strikingly, instead of the normal scattered pattern of ciliated cells in the skin, a majority of the cells in the skin were multiciliated (FIG. 4C). This phenotype is not simply due to disabled Notch signaling, as the propagation of multiciliate cells also occurred in embryos co-injected with activated Notch ICD (Coffman et al. (1993) Cell 73:659). Furthermore, both layers of the ectoderm appear to form multiciliate cells in response to MCI, while only the inner cells does so when Notch is blocked (FIG. 6).

### 1Biv: An Inducible Form of MCI.

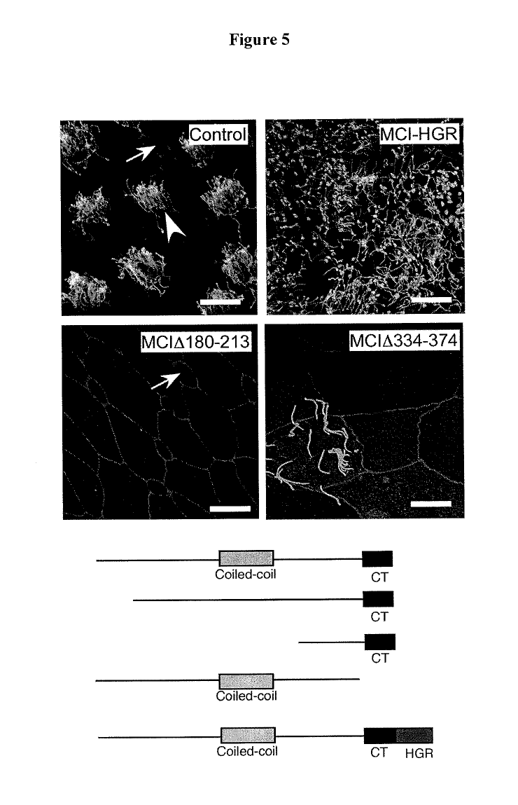

We next generated an inducible form of MCI by fusing it to the hormone-binding domain of the human glucocorticoid receptor (HGR) (Kolm et al. (1995) Dev. Biol. 171:267). Such fusion proteins have been extensively used to confer hormone inducibility onto transcription factors, as the fusion protein is retained in the cytoplasm, but can be moved to nucleus with the synthetic hormone, Dexamethasone (Dex). The fusion protein could also provide insight into whether MCI was acting cytoplasmically or in the nucleus. Indeed, when embryos were injected with MCI-HGR RNA and raised in absence of Dex, they developed normally, with a normal pattern of ciliated cell differentiation. However, when the same embryos were treated with Dex at the beginning of gastrulation, the same large-scale induction of multiciliate cell differentiation was observed as with the uninducible form of MCI (FIG. 5, top right). The results indicate that MCI acts in the nucleus, at least in part. The MCI-HGR construct allows for manipulation of MCI activity at different developmental stages.

### 1Bv: Dominant-Negative Mutants of MCI.

Several deletion mutants were generated to analyze the domain structure of MCI (FIG. 2). A form of MCI lacking the coiled-coil domain (MCIΔ180-213) not only failed to induce multiciliate cell differentiation or cell cycle arrest when expressed in embryos, it completely blocked the formation of multiciliate cells, thus behaving as a dominant-negative mutant (FIG. 5, bottom left). Again, as with the MCI morpholino phenotype, ciliated cells were missing based on cilia staining, as well as Centrin-GFP marked basal bodies. An even larger internal deletion of MCI (MCIΔ69-332, FIG. 2) also proved to be a potent inhibitor of ciliated cell differentiation. This deletion mutant lacks essentially all of the conserved regions of MCI except for the TIRT domain.

By contrast, the MCI mutant lacking the TIRT domain (MCIΔ334-374) produced a different phenotype from the internal deletions (FIG. 5, bottom right). This mutant failed to induce ectopic differentiation of normal-looking multiciliated cells. The MCIΔ334-374 mutant, however, did cause a striking increase in cell size, thus resembling the MCI phenotype, and while ciliated cells formed, many of them showed ciliogenesis defects not unlike those observed in FoxJ1 morphants. Thus, MCI lacking the TIRT domain still appears to cause cell cycle arrest, and acts weakly as a dominant-negative in terms of inhibiting ciliated cell differentiation. These data support the morpholino data by showing that MCI is required for multiciliate cell differentiation, and moreover, identify the coiled-coil and TIRT as functional domains.

### Example 5

### Generation of an MCI Antibody

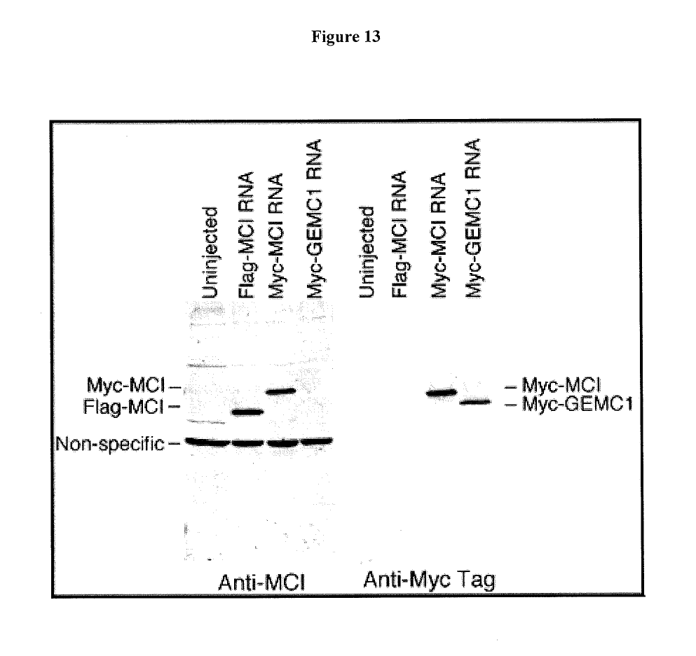

Xenopus embryos were injected with mRNA encoding Flag or Myc tagged MCI, Myc tagged GEMC1 or were uninjected. At stage 9, animal caps were removed and homogenized. Homogenates were then run out on a 10% SDS-PAGE gel. Blots with the anti-MCI antibody show specific bands at expected sizes for Flag and Myc tagged MCI but not GEMC1 (see FIG. 13, lanes 2, 3 and 4). The anti-myc tag blot shows that both Myc-MCI and Myc-GEMC1 are expressed (FIG. 13, lanes 7, 8).

### Example 6

### Mammalian MCI Induces Multiciliate Cells

We next turned to mammalian homologs of Xenopus MCI to confirm the expression and function of this protein. Indeed, we found that mammalian MCI is expressed in ciliated cells, and that mammalian MCI can induce multiciliate cell formation.

### MCI is Expressed in Ciliated Cells in Mouse Trachea

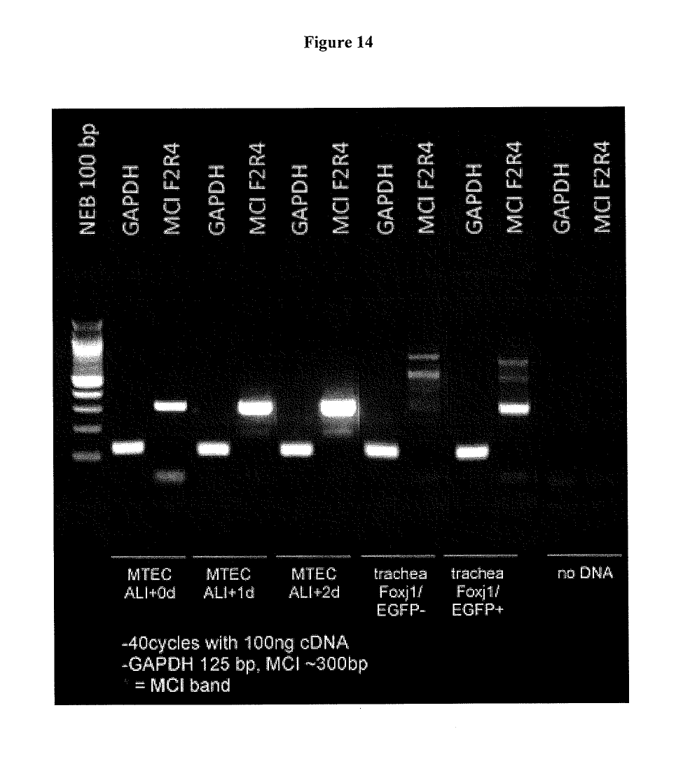

Mouse tracheal epithelial cell (MTEC) cultures were induced to differentiate into multiciliate cells by placing cultures at air-liquid interface (ALI) (FIG. 14). Tracheal epithelial cells can include goblet cells, which secrete mucus, and ciliated cells. Within 2 hours of the ALI exposure (ALI+0d), cultures expressed MCI and continue to express MCI through the first 2 days of differentiation (ALI+2d).

Tracheal cells from transgenic FoxJ1/EGFP mice (EGFP under the control of a FoxJ1 promoter) were isolated and FACS sorted. Only multiciliated cells expressed EGFP, and only EGFP+ cells expressed MCI (compare FIG. 14, lanes 9 and 11). The no DNA/negative control samples did not express MCI or the control gene GAPDH.

### Expression of Mammalian MCI Induces Xenopus Cells to Become Multiciliate

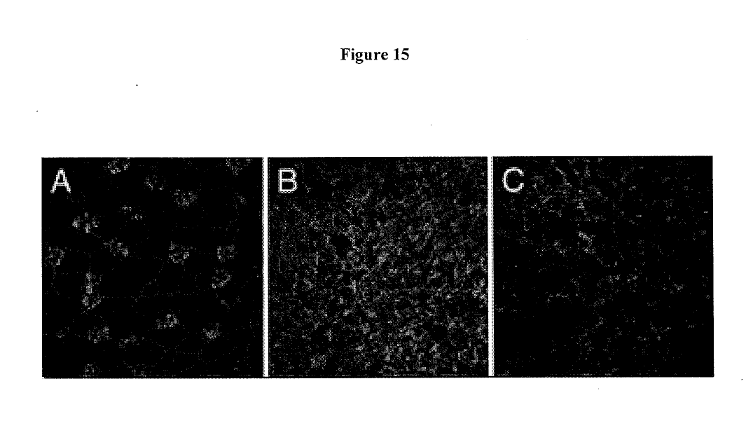

Xenopus embryos were injected with RNA encoding membrane RFP (mRFP) to mark the injected region along with Xenopus (FIG. 15B) or mouse (FIG. 15C) MCI. In embryos injected with mRFP alone (A) ciliated cells are evenly spaced (green staining). Upon injection of either Xenopus or mouse MCI almost all cells in the skin are ciliated. The results show that the mammalian MCI protein can actually act cross-species to function in Xenopus cells.

1. An isolated nucleic acid encoding a multicilin (MCI) protein.

2. The nucleic acid of claim 1, wherein said MCI protein comprises an amino acid sequence having at least 95% identity to a sequence selected from the group consisting of:
at least 40 consecutive amino acids of SEQ ID NO:2;
at least 40 consecutive amino acids of SEQ ID NO:4;
at least 40 consecutive amino acids of SEQ ID NO:5;
at least 40 consecutive amino acids of SEQ ID NO:6; and
at least 40 consecutive amino acids of SEQ ID NO:7.

3. The nucleic acid of claim 1, wherein said MCI protein comprises an amino acid sequence having at least 95% identity to a sequence selected from the group consisting of:
amino acids 178-215 of SEQ ID NO:2;
amino acids 161-227 of SEQ ID NO:2;
amino acids 178-374 of SEQ ID NO:2;
SEQ ID NO:2;
SEQ ID NO:4;
SEQ ID NO:5;
amino acids 172-238 of SEQ ID NO:6;
amino acids 172-379 of SEQ ID NO:6;
SEQ ID NO:6;
amino acids 176-242 of SEQ ID NO:7; and
SEQ ID NO:7.

4. The isolated nucleic acid of claim 1, wherein said nucleic acid is mammalian.

5. The isolated nucleic acid of claim 1, wherein said nucleic acid is human.

6. The isolated nucleic acid of claim 1, wherein said MCI protein comprises an amino acid sequence selected from the group consisting of:
SEQ ID NO:4;
SEQ ID NO:5;
SEQ ID NO:2;
SEQ ID NO:6; and
SEQ ID NO:7.

7-10. (canceled)

11. The isolated nucleic acid of claim 1, wherein said nucleic acid comprises a nucleic acid having at least 90% identity to the sequence of SEQ ID NO: 1.

12. An isolated expression vector comprising the nucleic acid of claim 1.

13. A transgenic cell comprising the expression vector of claim 12.

14. An isolated multicilin (MCI) protein.

15. The isolated MCI protein of claim 14, wherein said MCI protein comprises an amino acid sequence having at least 95% identity to a sequence selected from the group consisting of:
at least 40 consecutive amino acids of SEQ ID NO:2;
at least 40 consecutive amino acids of SEQ ID NO:4;
at least 40 consecutive amino acids of SEQ ID NO:5;
at least 40 consecutive amino acids of SEQ ID NO:6; and
at least 40 consecutive amino acids of SEQ ID NO:7.

16. The isolated MCI protein of claim 14, wherein the MCI protein comprises an amino acid sequence having at least 95% identity to a sequence selected from the group consisting of:
amino acids 178-215 of SEQ ID NO:2;
amino acids 161-227 of SEQ ID NO:2;
amino acids 178-374 of SEQ ID NO:2;
SEQ ID NO:2;
SEQ ID NO:4;
SEQ ID NO:5;
amino acids 172-238 of SEQ ID NO:6;
amino acids 172-379 of SEQ ID NO:6;
SEQ ID NO:6;
amino acids 176-242 of SEQ ID NO:7; and
SEQ ID NO:7.

17. The isolated MCI protein of claim 14, wherein said protein is mammalian.

18. The isolated MCI protein of claim 14, wherein said protein is human.

19. The isolated MCI protein of claim 14, wherein said MCI protein comprises an amino acid sequence selected from the group consisting of:
SEQ ID NO:4;
SEQ ID NO:5;
SEQ ID NO:2;
SEQ ID NO:6; and
SEQ ID NO:7.

20-23. (canceled)

24. An isolated antibody that specifically binds to the MCI protein of claim 1.

25. The antibody of claim 24, wherein said antibody specifically binds to Xenopus MCI protein.

26. The antibody of claim 24, wherein said antibody specifically binds to mouse MCI protein.

27. The antibody of claim 24, wherein said antibody specifically binds to human MCI protein.

28. A method for preparing a recombinant, multicilin (MCI)-expressing cell, said method comprising:
(i) introducing a nucleic acid encoding an MCI protein to a cell; and
(ii) allowing expression of said MCI protein in the cell, thereby preparing a recombinant, MCI-expressing cell.

29. The method of claim 28, wherein said recombinant MCI-expressing cell is a ciliate cell.

30. The method of claim 29, wherein said ciliate cell is a multiciliate cell.

31. The method of claim 28, wherein said recombinant cell divides to form a ciliate cell.

32. The method of claim 28, wherein said MCI protein comprises an amino acid sequence having at least 95% identity to a sequence selected from the group consisting of:
at least 40 consecutive amino acids of SEQ ID NO:2;
SEQ ID NO:4;
SEQ ID NO:5;
at least 40 consecutive amino acids of SEQ ID NO:6; and
at least 40 consecutive amino acids of SEQ ID NO:7.

33. A method for preparing a recombinant, multicellin (MCI)-expressing cell, said method comprising:
increasing the expression or activity of an endogenous MCI protein in a nonciliate cell, thereby preparing a recombinant, MCI-expressing cell.

34. The method of claim 33, wherein the recombinant, MCI-expressing cell is a ciliate cell.

35-45. (canceled)
